(19) United States
(12) Patent Application Publication
(10) Pub. No.: US 2017/0173297 A1
(43) Pub. Date: Jun. 22, 2017

(54) PILLOW AND METHOD OF CONTROLLING THE SAME

(71) Applicant: MOBILELAW, Daejeon (KR)

(72) Inventors: Rae Su PARK, Daejeon (KR); Gi Houn PARK, Daejeon (KR); Hyun Seung SHIN, Daejeon (KR); Kyung Tae KIM, Seoul (KR)

(21) Appl. No.: 15/311,603

(22) PCT Filed: May 26, 2016
(86) PCT No.: PCT/KR2016/005565

§ 371 Date: Nov. 16, 2016

(30) Foreign Application Priority Data
Jun. 2, 2015 (KR) 10-2015-0078229

(51) Int. Cl. A61M 21/00; G05B 19/048; A47G 9/10

(52) U.S. Cl. CPC A61M 21/00; A47G 9/1081; G05B 19/048; G05B 2219/24015; G05B 2219/23043

(57) ABSTRACT

A pillow may include a pillow form that includes a neck rest and a head rest, and a sleep controller that includes a body, at least one pair of vertically moving rods, and an actuating module configured to actuate the vertically moving rods.

### TECHNICAL FIELD

Embodiments relate to a pillow and a method of controlling the same.

### BACKGROUND ART

Sleep is the most basic desire of man. However, due to abuse of personal portable IT devices and watching of media being broadcast day and night, people have less time to sleep although they are tired from studying or hardworking during activity hours.

After all, people spend a lot of time to recover from fatigue during weekends, which causes an issue of irregular sleep.

Further, a temporary solution of using sleeping pills to treat sleep disorder or insomnia or going to facilities such as sleep clinics is not a fundamental solution, and may make such problems worse.

A variety of bedding that helps with recovery from fatigue and mental and physical relaxation of modern people lacking sleeping hours comes into the market. These products include beds, mattresses, and pillows with improved functionalities as significant devices.

However, a majority of manufactured pillows merely guide an average head position under the name of sleep science, and may not perfectly reflect different sleep positions or sleep habits of people and thus may cause secondary issues.

### DISCLOSURE OF INVENTION

### Technical Goals

An aspect provides technology that induces optimal sleep for a user by reflecting sleep positions or sleep habits of the user using a number of vertically moving rods.

Another aspect provides an environment that maintains deep sleep of a user while correcting bad sleep positions or sleep habits of the user.

### Technical Solutions

The above technical goals may be achieved by providing a pillow and a method of controlling the same which will be set forth hereinafter.

According to an aspect, there is provided a pillow including a pillow form that includes a neck rest and a head rest, and a sleep controller that includes a body, at least one pair of vertically moving rods, and an actuating module configured to actuate the vertically moving rods.

The pillow may further include an operator connected to the sleep controller.

A plurality of pillow form hollows may be formed in the neck rest, and the vertically moving rods may be disposed to penetrate through the pillow form hollows.

Hollows may be formed in internal portions of the vertically moving rods.

The vertically moving rods may include at least one pair of first rods, and at least one pair of second rods disposed parallel with the first rods.

At least one of the first rods and the second rods may be disposed slantly to form an acute angle based on a vertical state.

The acute angle may be in the range of 5 degrees to 20 degrees.

The first rods and the second rods may be configured to be vertically actuated separately.

The actuating module may include at least one of a cam gear, a rack and pinion gear, or a linear motor.

The sleep controller may further include a sleep controller cover configured to cover the body, the sleep controller cover including an elastic material, and rod covers configured to cover the vertically moving rods, the rod covers including an elastic material.

The sleep controller may further include a speaker.

The pillow may further include a pressure sensor configured to sense a state of a head or neck rested on the pillow.

The pillow may further include a gyro sensor configured to sense a position or angle of the pillow form.

The pillow may further include a thermo-hygro sensor configured to sense temperature and humidity of the pillow.

The pillow may further include a communicator configured to communicate with an external terminal.

The communicator may be configured to transmit user sleep information sensed by the sleep controller to the external terminal, and receive control information related to the sleep controller from the external terminal.

According to another aspect, there is also provided a method of controlling a pillow, the method including comparing left pressure and right pressure of a neck or head of a user, maintaining lateral balance of the neck or head of the user, and inducing sleep for the user.

The comparing may include measuring the left pressure and the right pressure through pressure sensors of vertically moving rods.

The maintaining may include moving the vertically moving rods vertically, measuring the left pressure and the right pressure to determine whether the balance is maintained, and repeating the moving when the balance is not maintained.

The method may further include storing positions of the vertically moving rods when the balance is maintained.

The inducing may include raising the vertically moving rods, maintaining heights of the raised vertically moving rods when the raising is suspended, and lowering the vertically moving rods.

The inducing may further include, after the lowering, maintaining heights of the lowered vertically moving rods when the lowering is suspended.

The raising, the maintaining of the heights of the raised vertically moving rods, the lowering, and the maintaining of the heights of the lowered vertically moving rods may be performed at intervals of an operation performance time, and may be performed at intervals of a changed operation performance time when a cycle performance time elapses.

At least one cycle performance time may be set, and the inducing may be performed during a preset total performance time.

The raising or the lowering may be performed with respect to the plurality of vertically moving rods sequentially.

The method may further include suspending an operation of the vertically moving rods when an abnormal operating state of the pillow is sensed.

The suspending may include determining that the user left the pillow when pressure applied to the pillow is not measured or decreases greatly, and suspending the operation of the vertically moving rods.

The suspending may include suspending the operation of the vertically moving rods when a horizontal degree of the pillow measured by a gyro sensor is outside of a preset range.

The suspending may include suspending the operation of the vertically moving rods when an impact is applied to the pillow.

The suspending may include suspending the operation of the vertically moving rods when noise is not sensed through a microphone.

### Effects

According to embodiments, a pillow and a method of controlling the same may induce optimal sleep for a user through reflection of sleep positions or sleep habits of the user by moving vertically moving rods based on a position of the user.

According to embodiments, a pillow and a method of controlling the same may provide an environment that maintains deep sleep of a user while correcting bad sleep positions or sleep habits of the user.

The effects of the present invention are not limited to the aforementioned effects and other effects not mentioned above will be clearly understood by those skilled in the art from the following description.

### BRIEF DESCRIPTION OF DRAWINGS

FIG. 1 is a perspective view of a pillow according to an embodiment.

FIG. 2 is an exploded perspective view of a pillow according to an embodiment.

FIG. 3 is an exploded perspective view of a sleep controller and a sleep controller cover according to an embodiment.

FIGS. 4A through 4C are side views illustrating arrangements of vertically moving rods according to an embodiment.

FIGS. 5A through 5D are top views illustrating arrangements of vertically moving rods according to an embodiment.

FIGS. 6A and 6B are views illustrating actuating modules according to an embodiment.

FIG. 7 is a view illustrating sensors according to an embodiment.

FIG. 8 illustrates a pillow being used according to an embodiment.

FIG. 9 is a flowchart illustrating a method of controlling a pillow according to an embodiment.

FIG. 10 is a flowchart illustrating an operation of maintaining lateral balance according to an embodiment.

FIG. 11 is a flowchart illustrating an operation of inducing sleep according to an embodiment.

FIG. 12 is a flowchart illustrating an operation of sensing an abnormal operating state according to an embodiment.

FIG. 13 illustrates a pillow and an external terminal according to an embodiment.

### BEST MODE FOR CARRYING OUT THE INVENTION

Hereinafter, embodiments will be described in detail with reference to the accompanying drawings. Regarding the reference numerals assigned to the elements in the drawings, it should be noted that the same elements will be designated by the same reference numerals, wherever possible, even though they are shown in different drawings. Also, in the description of embodiments, detailed description of well-known related structures or functions will be omitted when it is deemed that such description will cause ambiguous interpretation of the embodiments.

In addition, terms such as first, second, A, B, (a), (b), and the like may be used herein to describe components. Each of these terminologies is not used to define an essence, order or sequence of a corresponding component but used merely to distinguish the corresponding component from other component(s). It should be noted that if it is described in the specification that one component is “connected”, “coupled”, or “joined” to another component, a third component may be “connected”, “coupled”, and “joined” between the first and second components, although the first component may be directly connected, coupled or joined to the second component.

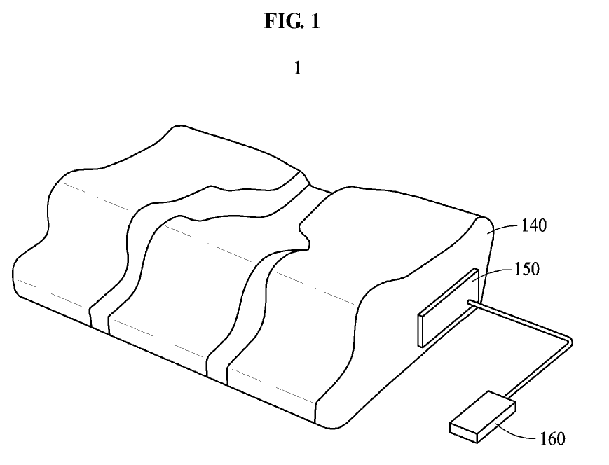

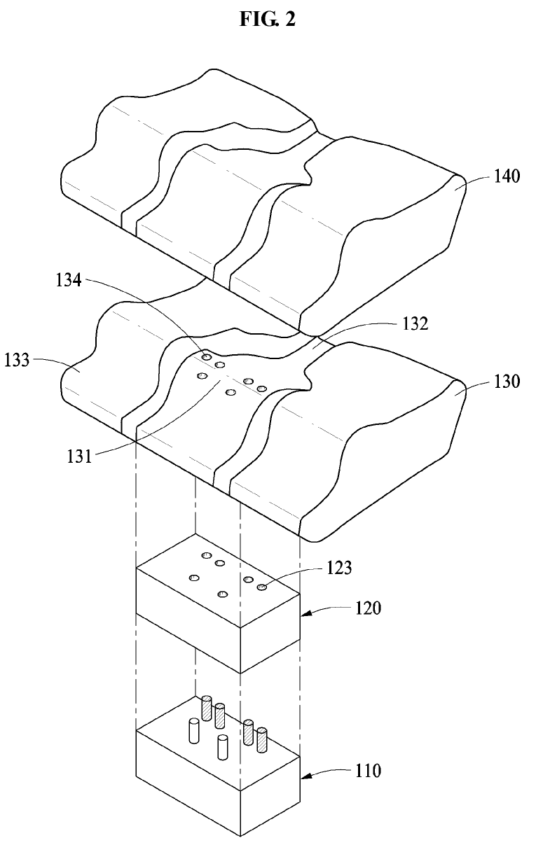

FIG. 1 is a perspective view of a pillow according to an embodiment, and FIG. 2 is an exploded perspective view of the pillow according to an embodiment.

Referring to FIGS. 1 and 2, a pillow 1 may induce optimal sleep for a user by reflecting sleep positions or sleep habits of the user through a vertically moving rod 112, 113 supporting a head or neck of the user.

The pillow 1 may correct bad sleep positions or sleep habits of the user, and provide an environment that may maintain deep sleep of the users. Hereinafter, descriptions will be provided in detail.

The pillow 1 may include a pillow form 130, and a pillow cover 140 configured to cover the pillow form 130.

The pillow form 130 may include a neck rest 131 and a head rest 132 that constitute a body 111 of the pillow 1.

The neck rest 131 and the head rest 132 may be concave, and support the neck and the head of the user, respectively. A height of the neck rest 131 may be greater than a height of the head rest 132.

However, the shapes of the neck rest 131 and the head rest 132 are not limited thereto. The shapes or arrangement thereof may vary in view of a body shape of the user.

The pillow form 130 may be configured in a shape corresponding to a shape of the user. For example, curves of the pillow form 130 may be formed based on shapes of the head and the neck of the user such that the head and the neck of the user may be rested to fit in the pillow form 130.

The pillow form 130 may include a deformable material to reflect a change caused by a difference in the head or neck of the user. Further, a shoulder support 133 may be provided on one side of the pillow form 130 to support shoulders of the user.

The pillow 1 is used in a state of being in contact with the user. Thus, the pillow cover 140 may be provided to prevent contamination of the pillow form 130. The pillow cover 140 may be configured in a shape corresponding to the pillow form 130 or may include an elastic material. A general pillow cover 140 may also be used. Thus, although the pillow cover 140 and the pillow form 130 are combined, the shapes of the neck rest 131, the head rest 132, and the shoulder support 133 may be maintained.

The pillow cover 140 may include an outer cover and an inner cover. For example, a portion of the outer cover may be mesh, and may include at least one of fabric, leather, or pure cotton material inside thereof. Further, the inner cover may include a pure cotton, waterproof, or water-repellent material.

However, the pillow cover 140 may also be configured as a single cover. The pillow cover 140 is not limited to the above example.

A sleep controller 110 may be provided in the pillow form 130. The sleep controller 110 may include the vertically moving rod 112, 113, and the vertically moving rod 112, 113 may support the head or neck of the user to induce deep sleep for the user.

A sleep controller cover 120 may be provided to cover the sleep controller 110. Cover hollows 123 may be formed in the sleep controller cover 120, and the vertically moving rods 112, 113 may penetrate through the cover hollows 123 such that the sleep controller cover 120 may be combined with the sleep controller 110.

Further, pillow form hollows 134 may be formed in the pillow form 130, and the vertically moving rods 112, 113 may penetrate through the pillow form hollows 134 such that the pillow form 130 may be combined with the sleep controller 110.

A function device 150 may be provided on one side of the pillow form 130. A microphone terminal, an earphone terminal, and a universal serial bus (USB) terminal may be provided in the function device 150. A power supply and the operator 160 configured to control the sleep controller 110 may be connected to the function device 150.

The function device 150 may be disposed on a side of the pillow form 130. However, the function device 150 may also be disposed on a front side or a rear side of the pillow form 130. The position and function of the function device 150 are not limited thereto.

Further, a portion of the pillow cover 140 may be open to expose the function device 150 externally.

The operator 160 may be connected to the sleep controller 110, and the user may control the sleep controller 110 through the operator 160. The operator 160 may provide functions such as start, stop, and timer settings. The operator 160 may be connected to the sleep controller 110 through the function device 150.

The sleep controller 110 may be provided in a form of a remote control. The sleep controller 110 may include a display to display operating information of the pillow 1.

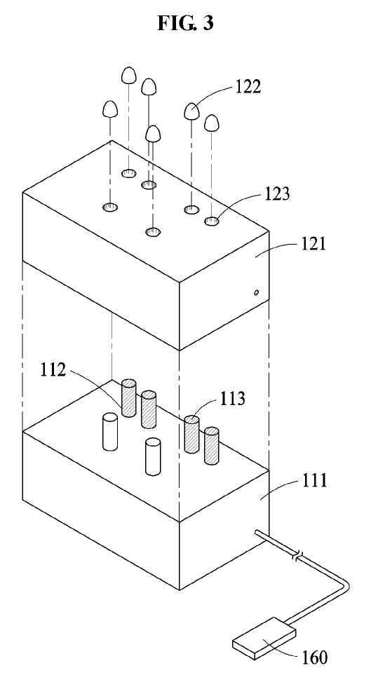

FIG. 3 is an exploded perspective view of a sleep controller and a sleep controller cover according to an embodiment.

Referring to FIG. 3, the sleep controller 110 may include the body 111, and the vertically moving rod 112, 113. At least one pair of vertically moving rods 112, 113 may be provided on the body 111.

The vertically moving rods 112, 113 may vertically move to pressurizing a body part of the user or change a state of the pillow form 130 supporting the user, thereby inducing deep sleep for the user. The vertically moving rods 112, 113 may operate separately or simultaneously.

A plurality of vertically moving rods 112, 113 may be disposed on both sides from a center of the body 111.

A plurality of pairs of vertically moving rods 112, 113 may be provided, and each pair may be disposed on both sides from the center of the body 111. A pair of vertically moving rods 112, 113 may be provided and disposed on both sides. Further, a plurality of pairs of vertically moving rods 112, 113 may be disposed to form a row or column.

For example, the vertically moving rod 112, 113 may include first rods 112 and second rods 113. At least one pair of first rods 112 may be provided, and the second rods 113 may be disposed parallel with the first rods 112.

At least one of the first rods 112 and the second rods 113 may be disposed slantly to from an acute angle based on a vertical state.

For example, the first rods 112 may be disposed perpendicular to the body 111, and the second rods 113 may be disposed slantly relative to a line perpendicular to the body 111 at 5 degrees to 20 degrees in one direction. Preferably, the slope of the second rods 113 may be 5 degrees to 15 degrees.

Hereinafter, for ease of description, a case in which the first rods 112 are disposed perpendicularly, and the second rods 113 are disposed slantly will be described as an example.

In view of a body structure of the user, the first rods 112 and the second rods 113 may be disposed. For example, the first rods 112 may support the neck of the user, and the slanted second rods 113 may support the head of the user.

The sleep controller cover 120 may cover and protect the sleep controller 110. For example, the sleep controller cover 120 may include an elastic material to closely protect the sleep controller 110 or absorb an impact applied to the sleep controller 110.

Rod covers 122 may be provided to cover and protect the vertically moving rods 112, 113. The rod covers 122 may include an elastic material.

For example, the sleep controller cover 120 and the rod covers 122 may be provided separately to cover the sleep controller 110 and the vertically moving rods 112, 113, respectively, or the sleep controller cover 120 and the rod covers 122 may be provided as an integral body.

The operator 160 may be connected to the sleep controller 110, and the user may control the sleep controller 110 using the operator 160.

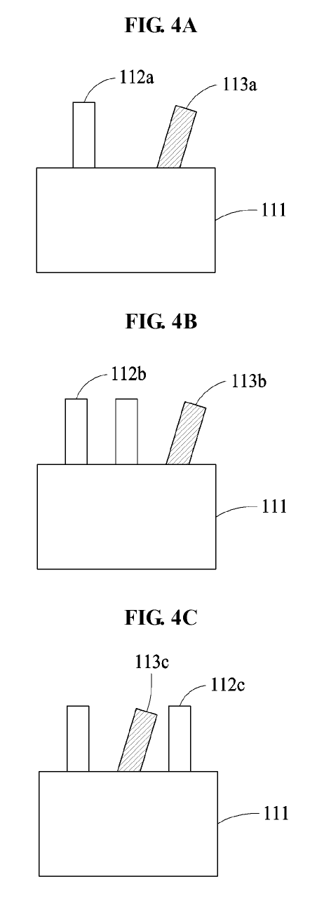

FIGS. 4A through 4C are side views illustrating arrangements of vertically moving rods according to an embodiment.

Referring to FIGS. 4A and 4C, the vertically moving rod 112, 113 may be provided in various arrangements in view of a purpose of the pillow 1 or the body structure of the user.

As shown in FIG. 4a, first rods 112a and second rods 113a may each be disposed in a single row. Thus, the first rods 112a may support the neck of the user, and the second rods 113a may support the head of the user.

As shown in FIG. 4B, first rods 112b may disposed in two rows and the second rods 113b may be disposed in a single row to increase an area supporting the neck of the user. As shown in FIG. 4C, first rods 112c may be disposed on both sides of the second rods 113c.

However, the arrangement of the vertically moving rod 112, 113 is not limited thereto.

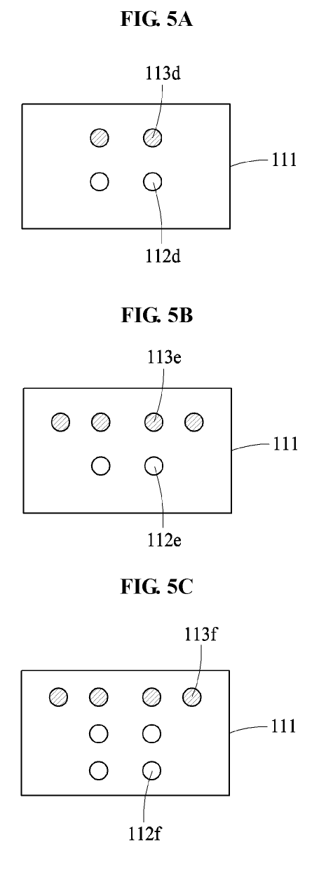

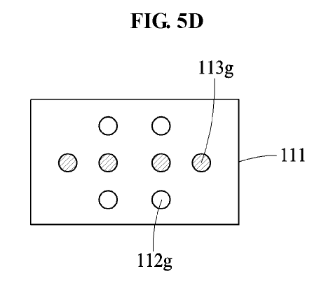

FIGS. 5A through 5D are top views illustrating arrangements of the vertically moving rod 112, 113 according to an embodiment.

Referring to FIGS. 5A through 5D, at least one pair of vertically moving rods 112, 113 may be disposed.

As shown in FIG. 5A, a pair of first rods 112d and a pair of second rods 113d may be provided. Each of the pair of first rods 112d and the pair of second rods 113d may be disposed on both sides from the center of the body 111.

As shown in FIG. 5B, a pair of first rods 112e and two pairs of second rods 113e may be disposed on both sides of the body 111. As shown in FIG. 5C, first rods 112f may be disposed in two rows and two pairs of second rods 113f may be disposed on both sides of the body 111.

As shown in FIG. 5D, two pairs of second rods 113g may be disposed in a row, and two pairs of first rods 112g may be disposed in two rows parallel with the second rods 113g.

However, the arrangement of the vertically moving rods 112, 113 is not limited thereto. That is, the arrangement of the vertically moving rods 112, 113 may vary in view of the purpose of the pillow 1 or the body structure of the user.

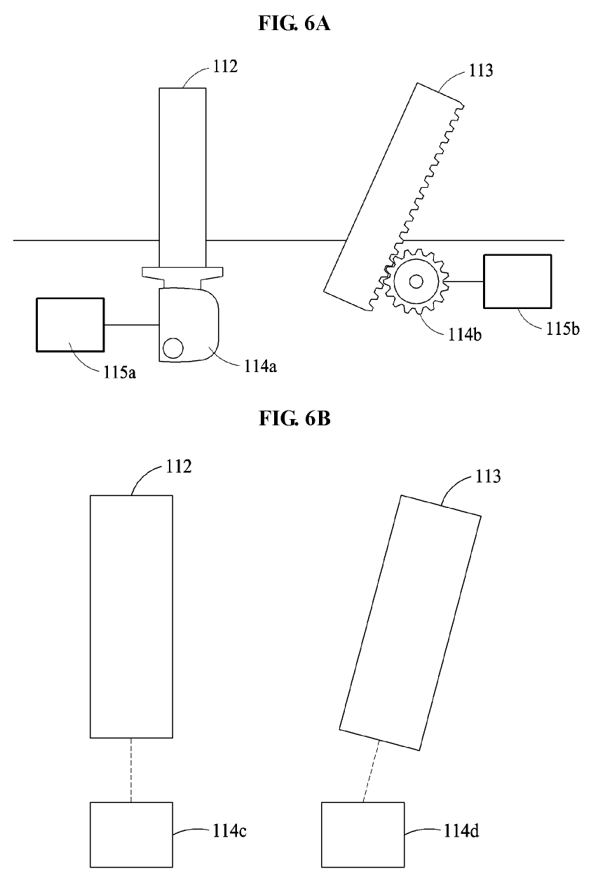

FIGS. 6A and 6B are views illustrating actuating modules according to an embodiment.

Referring to FIGS. 6A and 6B, the sleep controller 110 may include an actuating module configured to actuate the vertically moving rod 112, 113. The actuating module may include a gear 114a, 114b connected to vertically moving rod 112, 113, and a driving motor 115a, 115b.

The gear 114a, 114b may include a cam gear or a rack and pinion gear. For example, one of the cam gear and the rack and pinion gear may be used, or different types of gears may be used for the first rod 112 and the second rod 113.

As shown in FIG. 6A, the first rod 112 may be connected to the first gear 114a, and the first gear 114a may include a cam gear. The cam gear may have an eccentric rotation axis, thereby actuating the first rod 112 vertically. The first gear 114a may be connected to the first driving motor 115a, and the first driving motor 115a may provide driving force to the first gear 114a.

The second rod 113 may be connected to the second gear 114b, and the second gear 114b may include a pinion gear.

The second gear 114b may engage with a rack gear formed on the second rod 113 to actuate the second rod 113 vertically. The second gear 114b may be connected to the second driving motor 115b, and the second driving motor 115b may provide driving force to the second gear 114b.

As shown in FIG. 6B, the actuating module may include a gear structure using a linear motor. In this example, a first actuating module 114c and a second actuating module 114d may be connected directly to the first rod 112 and the second rod 113, respectively, to actuate the first rod 112 and the second rod 113 vertically.

However, the components and the operating method of the actuating module are provided as examples, and embodiments are not limited thereto.

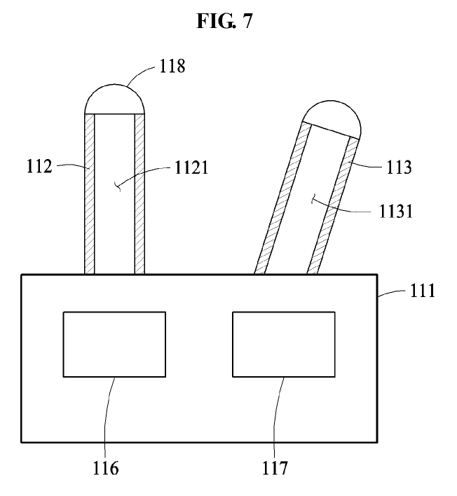

FIG. 7 is a view illustrating sensors according to an embodiment. Referring to FIG. 7, a pressure sensor 118 may be provided on one side of the first rod 112 or the second rod 113. For example, pressure sensors 118 may be disposed at end portions of the first rod 112 and the second rod 113 to sense pressure applied to a user.

The first rod 112 and the second rod 113 may include a first hollow 1121 and a second hollow 1131, respectively, and wires (not shown) may be disposed in the first hollow 1121 and the second hollow 1131 to connect the pressure sensor 118 and the sleep controller 110.

The pillow 1 may include a gyro sensor 116 configured to sense a position or angle of the pillow form 130, or a thermo-hygro sensor 117 configured to sense temperature and humidity.

The gyro sensor 116 may sense the position or angle at which the pillow form 130 is disposed to determine a disposition state of the pillow 1, and the thermo-hygro sensor 117 may sense surroundings or a state of the pillow 1 and provide the information to the user or control the sleep controller 110 based on the information.

Further, a speaker (not shown) may be provided on one side of the sleep controller 110, and the speaker may transfer operating information to the user or provide a warning sound.

A communicator 170 may be provided to communicate with an external terminal T. For example, the communicator 170 may transmit user sleep information sensed by the sleep controller 110 to the external terminal T, and receive control information related to the sleep controller 110 from the external terminal T.

The external terminal T may include a smart phone, a tablet computer, a control server, or a personal computer, and a communication scheme between the communicator 170 and the external terminal T is not limited.

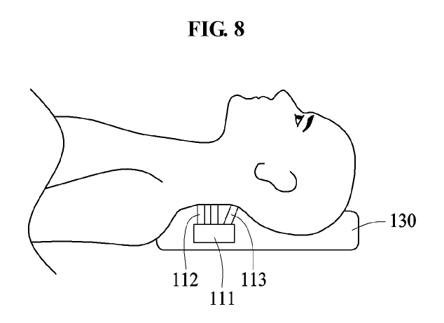

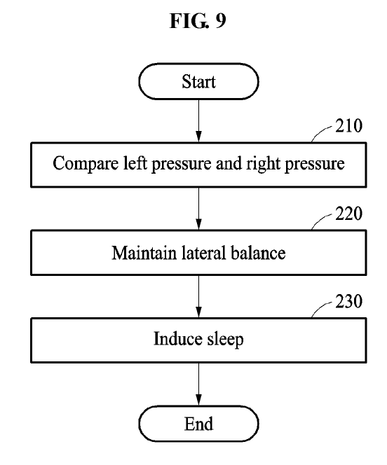

FIG. 8 illustrates a pillow being used according to an embodiment. Referring to FIG. 8, when a user rests a head on the pillow 1, the vertically moving rod 112, 113 may support or pressurize a body part of the user. For example, the first rod 112 disposed slantly may support the head of the user, and the second rods 113 disposed vertically may support a neck of the user.

However, in a case in which the first rods 112 and the second rod 113 support the user, the configuration or disposition thereof may vary based on a detailed purpose of the pillow 1 or the user.

FIG. 9 is a flowchart illustrating a method of controlling a pillow according to an embodiment.

Referring to FIG. 9, a method of controlling the pillow 1 may include operation 210 of comparing left pressure and right pressure applied by a user to the pillow 1, operation 220 of maintaining lateral balance, and operation 230 of inducing sleep.

Operation 210 may be performed by the pressure sensor 118 disposed on one side of the vertically moving rod 112, 113. When the user rests a head on the pillow 1, a neck or head of the user may be in contact with the vertically moving rod 112, 113, and an imbalanced state of the neck or head may be measured by the pressure sensor 118.

Operation 220 may be performed by moving the vertically moving rod 112, 113 vertically in response to sensing of the imbalanced state of the neck or head.

In operation 230, sleep of the user may be induced by moving at least one pair of vertically moving rods 112, 113 vertically. A speed at which the vertically moving rods 112, 113 are moved may be maintained at a stable speed not to interrupt sleep of the user.

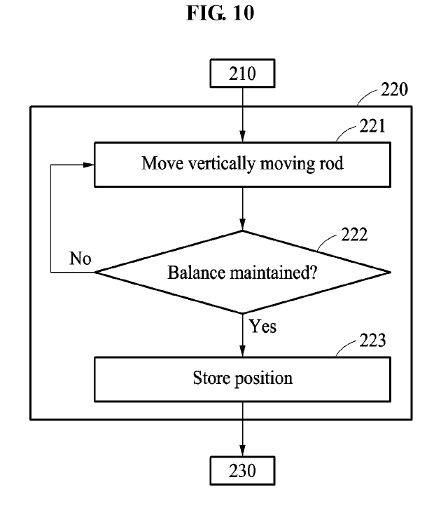

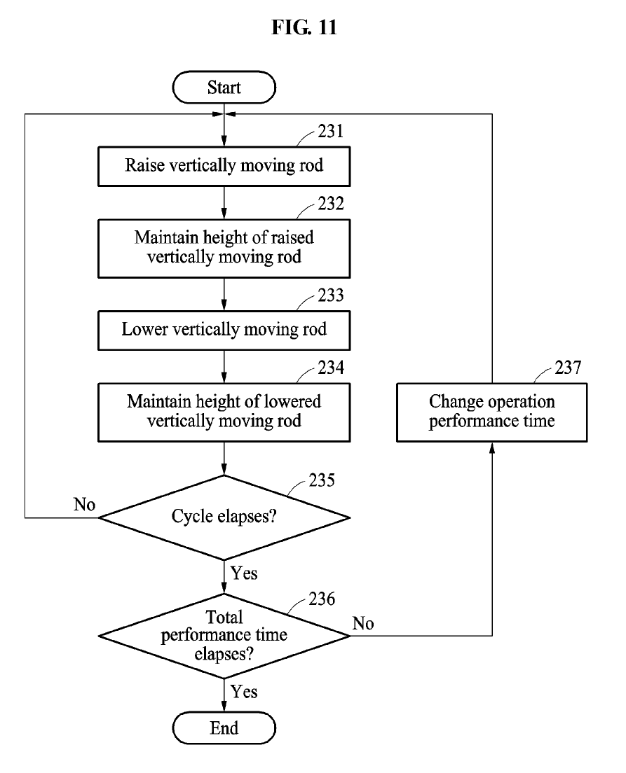

FIG. 10 is a flowchart illustrating an operation of maintaining lateral balance according to an embodiment.

Referring to FIG. 10, operation 220 of FIG. 9 may include operation 221 of moving the vertically moving rod 112, 113 vertically, operation 222 of repeating the moving, and operation 223 of storing a position.

In operation 220, the vertically moving rod 112, 113 may be moved based on pressure measured at the neck or head. For example, the vertically moving rod 112, 113 may be lowered at a portion at which pressure is relatively high, and the vertically moving rod 112, 113 may be raised at a portion at which pressure is relatively low. For example, balance may be achieved by comparing left pressure and right pressure of an occipital bone and a cervical vertebral tapping portion.

The left pressure and the right pressure may be measured and a change in the left pressure and the right pressure may be sensed. When balance of the neck or head is not maintained in operation 222, the moving of the vertically moving rod 112, 113 may be repeated. That is, when the left pressure and the right pressure of the neck or head are measured at imbalanced values, the vertically moving rod 112, 113 may be moved vertically, and the vertical movement may be suspended when lateral balance is achieved. The above operations may be performed at least one time.

When the balance of the neck or head is maintained, a position of the vertically moving rod 112, 113 may be stored and fixed, in operation 223.

FIG. 11 is a flowchart illustrating an operation of inducing sleep according to an embodiment.

Referring to FIG. 11, an operation of inducing sleep may include operation 231 of raising the vertically moving rod 112, 113, operation 232 of maintaining a height of the raised vertically moving rod 112, 113, and operation 233 of lowering the vertically moving rod 112, 113. The operation of inducing sleep may further include operation 234 of maintaining a height of the lowered vertically moving rod 112, 113. That is, the vertically moving rod 112, 113 may be moved through four operations of raising, maintaining the raised rod height, lowering, and maintaining the lowered rod height.

The vertically moving rod 112, 113 may be moved vertically at a preset speed to induce sleep of the user. A vertical movement range of the vertically moving rod 112, 113 may be set not to interrupt sleep of the user. For example, the vertically moving rod 112, 113 may be raised in a range of 0.5 cm to 3 cm, preferably, about 1.2 cm.

The raising, maintaining the raised rod height, lowering, and maintaining the lowered rod height of the vertically moving rod 112, 113 may be performed sequentially. For example, vertically moving rods 112, 113 disposed in a second row may be raised after vertically moving rods 112, 113 disposed in a first row are raised, or the vertically moving rods 112, 113 disposed in the second row may be lowered after the vertically moving rods 112, 113 disposed in the first row are lowered.

The raising, maintaining the raised rod height, and lowering of the vertically moving rod 112, 113 may be performed at predetermined time intervals.

For example, an operation performance time may be set to 5 seconds. In detail, the vertically moving rod 112, 113 may be raised slowly for 5 seconds, the height of the raised vertically moving rod 112, 113 may be maintained for 5 seconds, and the vertically moving rod 112, 113 may be lowered slowly for 5 seconds. Then, the height of the lowered vertically moving rod 112, 113 may be maintained for 5 seconds.

In operation 235, the raising, maintaining the raised rod height, lowering, and maintaining the lowered rod height of the vertically moving rod 112, 113 may be repeated during a preset cycle performance time. For example, when the cycle performance time is set to 5 minutes, the above operations may be repeated for 5 minutes. That is, for the 5 minutes being set, the raising, maintaining the raised rod height, lowering, and maintaining the lowered rod height of the vertically moving rod 112, 113 may be performed at intervals of 5 seconds.

In operation 237, the operation performance time may be changed when the cycle performance time elapses.

For example, when 5 minutes elapse, the operation performance time may be changed to 7 seconds. The raising, maintaining the raised rod height, lowering, and maintaining the lowered rod height of the vertically moving rod 112, 113 may be performed at intervals of 7 seconds. The raising, maintaining the raised rod height, lowering, and maintaining the lowered rod height of the vertically moving rod 112, 113 may be repeated for 5 minutes, and the operation performance time may be changed when the 5 minutes elapses.

Such repetition may be performed during a total performance time, in operation 236. When the total performance time elapses, sleep induction may be terminated.

That is, the sleep induction may be performed during the total performance time, and the total performance time may be selectively set to, for example, 10 minutes, 15 minutes, 30 minutes, 45 minutes, or 1 hour.

The total performance time may be divided into a number of cycle performance times. For example, when the total performance time is 30 minutes, cycle performance times may be set to 5 minutes, 10 minutes, 10 minutes, and 5 minutes. The cycle performance times may refer to a start cycle, a sleep induction cycle, and a cycle to ease the user.

The operation performance time may be set for each cycle performance time. For example, the operation performance time may be set to 5 seconds, 7 seconds, or 10 seconds. Based on the operation performance time, a time to perform each of the raising, maintaining the raised rod height, lowering, and maintaining the lowered rod height of the vertically moving rod 112, 113 may be determined.

However, the time settings described above are provided as examples only. Operation 230 is not limited thereto. For example, the raising of the vertically moving rod 112, 113 may be performed for 5 seconds, the raised vertically moving rod 112, 113 may be maintained for 10 seconds, and the lowering of the vertically moving rod 112, 113 may be performed for 5 seconds. The cycle performance time or the total performance time may be set differently.

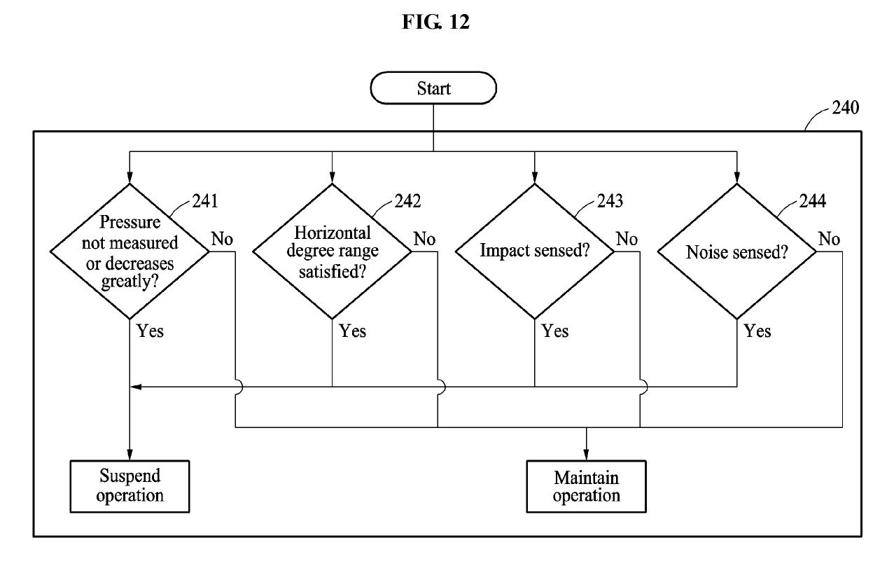

FIG. 12 is a flowchart illustrating an operation of sensing an abnormal operating state according to an embodiment.

Referring to FIG. 12, operation 240 of suspending an operation of the vertically moving rod 112, 113 when an abnormal operating state of the pillow 1 is sensed may be performed during any operation of the method of controlling the pillow 1, and may be performed frequently.

In operation 240, an abnormal operating state of the pillow 1 may be sensed, and the operation of the vertically moving rod 112, 113 may be suspended when the abnormal state is sensed.

For example, when pressure applied to the pillow 1 is not measured or decreases greatly in operation 241, it may be determined that the user left the pillow 1, and thus the operation of the vertically moving rod 112, 113 may be suspended.

That is, when the pressure is not measured by the pressure sensor 118 or decreases greatly as the user left the pillow 1 or suddenly changed a position, it may be determined to be an abnormal operation and the operation of the vertically moving rod 112, 113 may be suspended.

When a position or angle of the pillow 1 measured by the gyro sensor 116 is outside of a preset range in operation 242, the operation of the vertically moving rod 112, 113 may be suspended. For example, when the position or angle measured by the gyro sensor 116 is out of an ordinary state, it may be determined that the pillow 1 is out of the position or moves, the operation of the vertically moving rod 112, 113 may be suspended.

When an impact is applied to the pillow 1 in operation 243, the operation of the vertically moving rod 112, 113 may be suspended. For example, when the user suddenly rests the head on the pillow 1 or the pillow 1 falls out of the bed, it may be determined to be an abnormal state, and thus the operation of the vertically moving rod 112, 113 may be suspended.

When noise is not sensed through a microphone in operation 243, the operation of the vertically moving rod 112, 113 may be suspended. For example, a snoring state of the user may be recognized through the microphone. When it is determined that the user is present, the vertically moving rod 112, 113 may be operated. When noise is not sensed through the microphone, it may be determined that the user left and thus the operation of the vertically moving rod 112, 113 may be suspended.

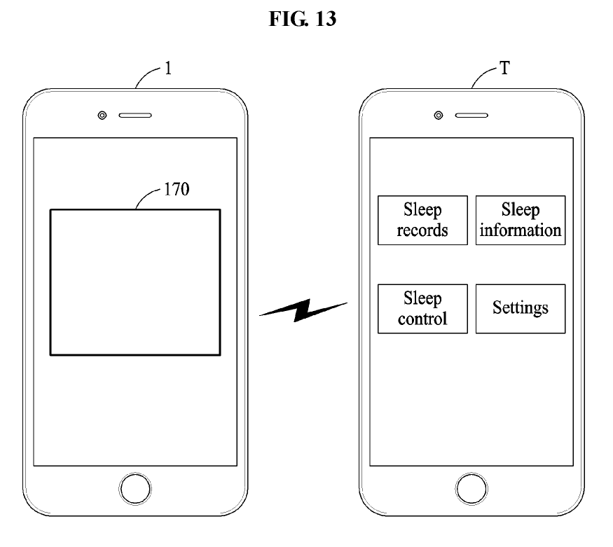

FIG. 13 illustrates a communication type between the pillow 1 and the external terminal T according to an embodiment. Referring to FIG. 13, the pillow 1 may include the communicator 170 to communicate with the external terminal T.

The external terminal T may include a smart phone, a tablet computer, a control server, or a personal computer. The communicator 170 may communicate with the external terminal T using a communication method such as Bluetooth or wireless-fidelity (Wi-Fi).

The external terminal T may receive user information being input and transfer the user information to the pillow 1, or display operating information transmitted from the pillow 1 or the user information to a user.

For example, an application for operation of the pillow 1 may be installed in the external terminal T, and user information such as a date of birth, a gender, and an age of the user may be input through the application.

Additionally, intake information related to food or alcohol, exercise information such as an exercise type or exercise quantity, and user state information such as fatigue or comfort may be input through the application. Through interoperation with the application which records exercise information, exercise information of the user may be received.

The external terminal T may provide information such as biorhythms or Five Motions and Six Atmospheres may be provided the above information, and provide the user with temperature, humidity, snoring noise, sleeping hours, or sleep state information sensed through the pillow 1.

Further, the external terminal T may provide information related to sleeping hours, sleep motions, sleep cycle analysis, and periodical sleep analysis of the user measured from the pillow 1, and provide the user with information related to recommended sleeping hours through a comparison with average sleeping hours for ages or genders, recommended sleeping hours corresponding to input information, or recommended sleeping hours corresponding to measured information.

The operation of the pillow 1 may be initiated or suspended through the external terminal T, and control information may be transmitted to the pillow 1 by setting a total performance time, a cycle performance time, and an operation performance time. That is, the external terminal T may also perform the function of the operator 160.

The pillow and the method of controlling the same described above may induce optimal sleep for a user by reflecting sleep positions or sleep habits of the user using a number of vertically moving rods, and provide an environment that maintains deep sleep of the user while correcting bad sleep positions or sleep habits of the user.

A number of examples have been described above. Nevertheless, it should be understood that various modifications may be made. For example, suitable results may be achieved if the described techniques are performed in a different order and/or if components in a described system, architecture, device, or circuit are combined in a different manner and/or replaced or supplemented by other components or their equivalents.

Accordingly, other implementations are within the scope of the following claims.

1.-30. (canceled)

31. A pillow, comprising:
a pillow form that comprises a neck rest and a head rest; and
a sleep controller that comprises a body, at least one pair of vertically moving rods, and an actuating module configured to actuate the vertically moving rods.

32. The pillow of claim 31, further comprising:
an operator connected to the sleep controller.

33. The pillow of claim 31, wherein a plurality of pillow form hollows are formed in the neck rest, and
the vertically moving rods are disposed to penetrate through the pillow form hollows.

34. The pillow of claim 31, wherein hollows are formed in internal portions of the vertically moving rods.

35. The pillow of claim 31, wherein the vertically moving rods comprise:
at least one pair of first rods; and
at least one pair of second rods disposed parallel with the first rods.

36. The pillow of claim 35, wherein at least one of the first rods and the second rods is disposed slantly to form an acute angle based on a vertical state.

37. The pillow of claim 36, wherein the first rods and the second rods are configured to be vertically actuated separately.

38. The pillow of claim 31, wherein the actuating module comprises at least one of a cam gear, a rack and pinion gear, or a linear motor.

39. The pillow of claim 31, wherein the sleep controller further comprises:
a sleep controller cover configured to cover the body, the sleep controller cover including an elastic material; and
rod covers configured to cover the vertically moving rods, the rod covers including an elastic material.

40. The pillow of claim 1, further comprising:
a communicator configured to communicate with an external terminal,
wherein the communicator is configured to transmit user sleep information sensed by the sleep controller to the external terminal, and receive control information related to the sleep controller from the external terminal.

41. A method of controlling a pillow, the method comprising:
comparing left pressure and right pressure of a neck or head of a user;
maintaining lateral balance of the neck or head of the user; and
inducing sleep for the user.

42. The method of claim 41, wherein the comparing comprises measuring the left pressure and the right pressure through pressure sensors of vertically moving rods.

43. The method of claim 41, wherein the maintaining comprises:
moving the vertically moving rods vertically;
measuring the left pressure and the right pressure to determine whether the balance is maintained; and
repeating the moving when the balance is not maintained.

44. The method of claim 41, further comprising:
storing positions of the vertically moving rods when the balance is maintained.

45. The method of claim 41, wherein the inducing comprises:
raising the vertically moving rods;
maintaining heights of the raised vertically moving rods when the raising is suspended; and
lowering the vertically moving rods.

46. The method of claim 45, the inducing further comprises:
after the lowering,
maintaining heights of the lowered vertically moving rods when the lowering is suspended.

47. The method of claim 46, wherein the raising, the maintaining of the heights of the raised vertically moving rods, the lowering, and the maintaining of the heights of the lowered vertically moving rods are performed at intervals of an operation performance time, and are performed at intervals of a changed operation performance time when a cycle performance time elapses.

48. The method of claim 45, wherein the raising or the lowering is performed with respect to the plurality of vertically moving rods sequentially.

49. The method of claim 41, further comprising:
suspending an operation of the vertically moving rods when an abnormal operating state of the pillow is sensed.

50. The method of claim 49, wherein the suspending comprises determining that the user left the pillow when pressure applied to the pillow is not measured or decreases greatly, and suspending the operation of the vertically moving rods.
